(19) United States
(12) Patent Application Publication
(10) Pub. No.: US 2003/0190290 A1
(43) Pub. Date: Oct. 9, 2003

(54) Pharmaceutical compositions

(76) Inventor: Calvin Ross, Hants (GB)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/312,200

(22) PCT Filed: Jun. 22, 2001
(86) PCT No.: PCT/GB01/02761

(30) Foreign Application Priority Data
Jun. 22, 2000 (GB) 0015360.1
Jan. 23, 2001 (GB) 0101744.1

(51) Int. Cl. A61L 9/04; A61K 31/485; A61K 31/445

(52) U.S. Cl. 424/45; 514/282; 514/317

(57) ABSTRACT

The present invention relates to compositions and dispensing devices for improved administration of fentanyl and other opioid analgesics, such as alfentanil, carfentanil, remifentanil, sufentanil, buprenorphine, morphine, diamorphine, and the like.

The present invention relates to compositions and dispensing devices for improved administration of fentanyl and other opioid analgesics, such as alfentanil, carfentanil, remifentanil, sufentanil, buprenorphine, morphine, diamorphine, and the like.

Opioid analgesics are frequently used for the relief of moderate to severe pain, as well as in anaesthesia. The present invention relates primarily to the use of fentanyl and other opioid analgesics in pain management and in particular to the treatment of acute pain or “break-through” pain. Ideally, this type of pain relief has rapid onset. Fentanyl and other opioid analgesics have rapid effect following administration, making them particularly suited for the treatment of break-through pain. Nevertheless, onset of their analgesic effect can be slowed considerably if there is a delay between administration and uptake of the active agent into the blood. Such delay means that certain modes of administration are unsuitable for treatment requiring rapid onset and are therefore not to be used in the treatment of break-through pain.

A particularly important consideration when administering opioid analgesics is that the doses are accurately controlled and are reproducible. Firstly, it is imperative that the patient does not over-dose. Large doses of opioid analgesics may lead to respiratory depression and some euphoric activity, which can lead to abuse and dependency. Secondly, it is undesirable for the patient to be provided with a dose which is too small, as such a dose is likely to provide inadequate pain-relief.

Whilst the use of opioid analgesics, and especially fentanyl, for the treatment of pain has been proposed in the past, an inability to provide accurate and reproducible dosages severely reduces its actual use in practice.

The aim of the present invention is, therefore, to ensure that accurate and reproducible doses of formulations comprising opioid analgesics are administered to a patient. This is achieved by a combination of two features. Firstly, a mode of administration is selected which not only provides rapid absorption of a dispensed dose into the blood stream, but also provides absorption of a predictable amount of the dispensed opioid analgesic into the blood stream. Secondly, a formulation is provided which can be dispensed so that an accurate and reproducible amount of the opioid analgesic is present in each dispensed dose.

The present invention is particularly concerned with providing patient-controlled analgesia, where the patients self-administer the drug in response to the pain they are experiencing. This can reduce the period of time during which the patient's pain goes untreated. Ideally, for such self-pain management to be effective, the drugs must be available to the patient in such a way that they can be easily and safely administered.

A number of methods of administering fentanyl and other opioids are know, each having disadvantages which mean that the doses administered can be unpredictable and inaccurate. The known methods of administration include injection, inhalation (lung delivery), oral administration and transdermal absorption (using patches).

Oral administration of fentanyl is known, for example in the form of tablets, lozenges or elixirs and the like. Whilst this mode of administration is undoubtedly very convenient and easy for the patient, the active agent fentanyl is only slowly absorbed into the blood stream via the gastrointestinal tract and the onset of the drug's effect is therefore delayed. The drug also undergoes first-pass metabolism in the liver when administered orally. Furthermore, patients requiring break-through pain relief frequently drift in and out of consciousness and so administration of the analgesic in the form of a tablet or lozenge will therefore pose a risk of choking, making constant supervision of the patient essential.

Transdermal patches for the delivery of fentanyl are also known. They rely upon a gradual absorption of the active agent through the skin and have been designed for rate-controlled drug delivery, that is slow release over a long period of time. Transdermal patches provide a convenient mode of delivery for opioid analgesics over an extended period of time. Opioid analgesics were previously considered unsuitable for providing analgesic effect over a prolonged period of time because they exhibit rapid onset and have a relatively short period of analgesic effect. Despite the benefits of transdermal patches, the slow administration is clearly not suited to treatment of break-through pain, where immediate relief is desired. Transdermal patches have also been found to cause irritation in some patients. Studies have also shown that transdermal patches occasionally result in unexpectedly increased absorption and therefore severe toxicity.

Injection is obviously one mode of administration which will allow rapid onset of fentanyl and other opioid analgesics. However, injections are painful, especially when regular administration is required. It can also be difficult for patients to inject themselves, especially if weak or lacking co-ordination, often making it necessary for someone other than the patient to perform the administration. This clearly reduces the patient's own control over the dosage level.

An alternative means of administering fentanyl which provides rapid onset of the physiological effect is lung delivery by inhalation. Fentanyl aerosol compositions are known for inhalation, for example, using metered dose inhalers. Such inhalers represent a simple and portable means of providing on-demand dosing. The metered dose inhalers may be provided with safety mechanisms to ensure that the patient cannot administer too much drug over a certain period, thereby reducing the risk of an overdose. Aerosol compositions for inhalation are disclosed in WO90/07333 and WO95/31182. The compositions disclosed contain fentanyl or fentanyl derivatives, either in solution or suspension in the aerosol propellant system, optionally in the presence of a cosolvent.

However, there are some disadvantages associated with lung delivery. Inhalation of known formulations has been found to cause a pronounced and involuntary cough, possibly due to irritation of the trachea and lungs by the active agent in the composition. A further disadvantage associated with inhalation is that it requires patient coordination to properly draw the composition into the lungs. This means that failure to properly coordinate spraying the composition and inhaling may lead to only part of the dose reaching the lungs and being taken up. Additionally, the inhalation mode of administration also relies upon the patient's ability to inhale deeply and this can pose a problem where the patient is weak or a smoker, for example. The patency of the patient's airways is clearly also an important factor. Consequently, it is difficult to guarantee a predictable or consistent dose when administering the opioid formulation by inhalation.

It is probably as a result of the abovementioned problems associated with aerosol inhalation that no form of fentanyl lung delivery has been marketed to date.

The applicants have found that the disadvantages experienced when using the known methods of administration discussed above may be overcome when fentanyl or other opioid analgesics are administered sublingually, preferably as an aerosol spray. Sublingual delivery of a pharmaceutically active agent results in fast uptake. The active agent is administered to the oral and sublingual mucosa, from which it is rapidly absorbed into the bloodstream. Sublingual delivery also avoids first-pass metabolism of the active agent.

Sublingual spray delivery is preferred over other types of sublingual delivery such as tablets, lozenges and liquids, as the amount of the active agent which is accidentally swallowed and therefore is not immediately effective is significantly reduced. Additionally, the spray administration can result in even faster absorption of the active agent through the sublingual mucosa.

The sublingual spray mode of delivery does not suffer from the disadvantages discussed above in connection with inhalation. Rather, the amount of the dispensed dose which actually enters the blood stream can be accurately predicted, as only a small proportion of the dispensed dose will be “lost”, i.e. will not be absorbed through the sublingual mucosa.

Sublingual delivery of fentanyl and other opioid analgesics is clearly an attractive method of administration. For example, spraying the composition under the tongue is painless and much more suitable for self-administration for the majority of patients than injection. Furthermore, unlike lung delivery by inhalation, sublingual delivery does not cause irritation of the trachea and lungs and there is no attendant cough. A further advantage of sublingual delivery over lung delivery by inhalation is that there is no requirement for patient co-ordination or the ability to inhale deeply, the sprayed dose merely has to be directed under the tongue.

Aerosol formulations comprising fentanyl and other opioid analgesics are known in the prior art for administration by inhalation. The formulations disclosed generally comprise an opioid salt, a propellant and a solvent. As opioid salts are not soluble in propellants and are also not soluble in commonly used solvents, such as ethanol, these formulations are suspensions.

Suspension formulations have disadvantages which result in dispensed doses containing an unpredictable amount of the opioid analgesic, thereby rendering them unsuitable for providing the desired accurate and reproducible doses.

Opioid salts are soluble in water. However, aqueous solutions are not suitable for the purposes of the present invention, as they would require the inclusion of preservatives, which is undesirable in a pharmaceutical preparation. Indeed, aqueous pharmaceutical solutions are generally avoided because of their frequent instability.

In order for an accurate and reproducible amount of the opioid analgesic to be dispensed in a single dose, the opioid must be uniformly distributed throughout the formulation. If the formulation is a suspension, the opioid will not be uniformly distributed. It is well known to agitate a suspension formulation prior to dispensing, in order to disperse the suspended active agent. However, rapid creaming, settling or flocculation after such agitation are common sources of dose irreproducibility in suspension formulations.

In contrast, in a solution, the opioid analgesic would be uniformly dispersed, leading to the same amount of opioid always being present in a given volume of the formulation.

A further problem associated with the use of an aerosol suspension formulation is that the formulation is significantly more likely to clog the nozzle of the dispensing device than a solution is. Such clogging can, once again, render it very unlikely that accurate and reproducible doses are dispensed.

In contrast, to the opioid analgesic salts, the free base form of opioid analgesics are soluble in conventional propellants. Thus, formulations comprising an opioid analgesic free base will be solutions.

In accordance with a first aspect of the present invention, a pharmaceutical composition is provided comprising a solution of an opioid analgesic and a propellant, for sublingual administration. The opioid analgesic may be selected from fentanyl, alfentanil, carfentanil, remifentanil, sufentanil, buprenorphine, morphine, and diamorphine.

In a preferred embodiment of the invention, the opioid analgesic is in the free base form. Preferably, the opioid is fentanyl free base.

In another preferred embodiment, the composition is a solution which is substantially free of water. The concentration of water in the composition should be less than 0.25% and is preferably less than 0.15%.

The propellant may be, for example, 1,1,1,2-tetrafluoroethane (HFC-134a), 1,1,1,2,3,3,3-heptafluoropropane (HFC-227) or a combination thereof. An alternative propellant which may be used is butane.

In the past, aerosol compositions frequently included one or more chlorofluorocarbon as a propellant, dichloro-difluoromethane being commonly used. It is well documented that chlorofluorocarbons are implicated in the depletion of the ozone layer and their production, therefore, is being phased out. 1,1,1,2-tetrafluoroethane (HFC-134a) and 1,1,1,2,3,3,3-heptafluoropropane (HFC-227) are significantly less harmful to the ozone layer and they are of low toxicity and of suitable vapour pressure for use as aerosol propellants, making then suitable for use in pharmaceutical aerosols. An additional benefit is that HFC-134a and HFC-227 can be used in combination with many pharmaceutically active agents, without causing any degradation to them or reducing their physiological activity. They are also not flammable.

In a preferred embodiment, the composition according to the first aspect of the invention comprises between 50 and 99% w/w HFC-134a or HFC-227. More preferably, the amount of propellant is between 75 and 95% w/w.

Whilst the composition may comprise just the active agent and propellant, the propellant will evaporate upon spraying and the active agent will therefore have no other constituent to “hold it in place” at the sublingual mucosa. It is essential that the active agent remain in contact with the sublingual mucosa for long enough to allow absorption. It is therefore preferable for the composition of the present invention to include a carrier. In a preferred embodiment of the invention, the carrier is a lower alkyl (C₁-C₄) alcohol, a polyol, or a (poly) alkoxy derivative. In embodiments, the carrier is a C₁-C₄ alkyl alcohol or a lanolin alcohol and, preferably, is ethanol or isopropyl alcohol. The most preferred alcohol is ethanol. The carrier does not act as a solvent or co-solvent.

The preferred polyols include propylene glycol and glycerol and the preferred (poly) alkoxy derivatives include polyalkoxy alcohols, in particular 2-(2-ethoxyethoxy) ethanol (available under the Trademark Transcutol®).

Further preferred (poly)alkoxy derivatives include polyoxyalkyl ethers and esters, such as polyoxyethylene ethers or esters. The preferred polyoxyethylene ethers and esters are polyoxyethylene alkyl ethers, polyoxyethylene castor oil derivatives, polyoxyethylene sorbitan fatty acid esters and polyoxyethylene stearates.

The preferred fatty acid alkyl esters are ethyl oleate, isopropyl myristate and isopropyl palmitate. The preferred polyalkylene glycol is polyethylene glycol.

In preferred embodiments, the inventive composition can comprise up to 50% or, preferably, 25% w/w carrier. More preferred embodiments include between 3% and 15% w/w, or between 4 and 10% w/w carrier. The pharmaceutical compositions can comprise between 50% and 99% w/w, preferably between 75% and 99% w/w, and, more preferably, between 88% and 95% w/w HFC-134a or HFC-227.

In further embodiments, compositions used in the present invention can comprise a plurality of different carriers.

Further excipients can be included in the compositions employed in the present invention. For example, neutral oils as well as surfactants (the latter for aiding the smooth operation of the valve), as are well known to those skilled in the art, may be included.

Thus, in further preferred embodiments, compositions employed in the invention can comprise an organic surfactant. The preferred organic surfactant is oleyl alcohol, although others can be employed, including sorbitan trioleate, sorbitan mono-oleate, sorbitan monolaurate, polyoxyethylene (20) sorbitan monolaurate, polyoxyethylene (20) sorbitan mono-oleate, natural lecithin, oleyl polyoxytheylene (2) ether, stearyl polyoxyethylene (2) ether, lauryl polyoxyethylene (4) ether, block copolymers of oxyethylene and oxypropylene, oleic acid, synthetic lecithin, diethylene glycol dioleate, tetrahydrofurfuryl oleate, ethyl oleate, isopropyl myristate, glyceryl mono-oleate, glyceryl monostearate, glyceryl monoricinoleate, cetyl alcohol, stearyl alcohol, cetyl pyridinium chloride, olive oil, glyceryl monolaurate, corn oil, cotton seed oil or sunflower seed oil.

It is preferable to include a flavouring oil in a composition to be delivered sublingually, in order to mask the unpleasant taste of the combination of propellant and opioid analgesic. The preferred flavouring oil is peppermint oil, although it is clear that other flavour oils may be used, according to preference.

The inclusion of the flavouring oil is also beneficial as it serves to lubricate the valve of the dispensing device, thereby helping its smooth and reliable operation. Thus, inclusion of the flavouring oil obviates the need for the composition to include an additional lubricant.

Furthermore, a volatile oil, such as peppermint oil, acts as a penetration enhancer when included in a composition for aerosol sublingual delivery, thereby increasing the absorption of the active agent at the sublingual mucosa and making the administration more effective. Thus, inclusion of a volatile flavouring oil will further obviate the need for the composition to include an additional penetration enhancer.

In a particularly preferred embodiment of the invention, the composition a solution of fentanyl free base, ethanol as a carrier, and HFC-134a as a propellant.

According to a further preferred embodiment, the compositions of the present invention comprise opioid analgesics in combination with other pharmaceutically active agents. For example, the muscular rigidity, which may be experienced upon administration of fentanyl, may be antagonised by co-administration of a muscle relaxant, such as naxalone. Similarly, the respiratory depression which may be associated with the administration of opioid analgesics may be countered by administering an additional active agent.

In accordance with a second aspect of the present invention, devices are provided for sublingually delivery of the opioid analgesic compositions according to the first aspect of the invention. These devices can assist dispensing of accurate and reproducible doses, and/or can assist direction of the dispensed dose to the sublingual area, to ensure that as mush of the dispensed dose as possible is absorbed through the sublingual mucosa.

Devices for administering metered aerosol doses of pharmaceutical preparations are well known in the art, and include devices with inverted valves and upright valves. Devices with inverted valves are preferred, as the volumes of pharmaceutical composition that they dispense are more accurate and consistent. Examples of suitable upright valve devices for dispensing the pharmaceutical compositions described herein are readily available from Bespak PLC of Bergen Way, Kings Lynn, Norfolk PE30 2JJ, United Kingdom.

Devices with upright valves include those disclosed in WO 92/11190, U.S. Pat. No. 4,819,834 and U.S. Pat. No. 4,407,481. Many of these devices include metering valves having components formed from plastic materials, such as the valves available from Bespak PLC of Bergen Way, Kings Lynn, Norfolk PE30 2JJ, United Kingdom, in which the valve core, metering chamber and some other structural components are formed from plastic materials. The plastic materials currently used for forming these structural parts in valves employed with many chlorofluorocarbon containing compositions include certain acetal co-polymers.

Although the plastics employed to manufacture metering valves, including the aforementioned acetal co-polymers, have also been found to be stable in the presence of HFC-134a alone, the applicants, to their surprise, have determined that many of these plastics materials can be caused to swell in the presence of compositions which include certain carriers or active agent solubilising co-solvents with HFC-134a. When such swelling takes place in a valve, the fit of mutually slidable components, such as metering chambers and valve cores, is adversely effected and they can bind together or become loose, causing the valve to leak or cease functioning altogether. It is very important to avoid this problem in connection with the present invention.

This problem has now been solved by using a device for providing pharmaceutical doses comprising a container, filled with a pharmaceutical composition according to the first aspect of the invention, and valve means arranged for delivering aerosol doses of said pharmaceutical composition to the exterior of the container, wherein at least a portion of the device is formed from a polyester. Preferably, the valve means includes at least one component formed from a polyester, which component, more preferably, is a metering chamber and/or a valve core.

In further embodiments, the container comprises a polyester and, preferably, consists of metal lined with a polyester. The canister cap can also be so formed.

Apart from allowing the aforementioned swelling problem to be solved, an advantage of this aspect of the present invention is that use of expensive metal valve components can be avoided.

The preferred polyesters are polyalkylene benzene dicarboxylates, more preferably polyalkylene terephthalates and, most preferably, a polybutylene terephthalate.

Such materials, preferably, have a density of about 1.3g/cm³ and a water absorption of about 0.6% (23° C. saturation). The polyesters, also, are preferably partially crystalline in nature and have a crystalline melting range of 220-225° C.

Examples of suitable polybutylene terephthalates include those available under the Trademark Celanex® from Hoechst UK Limited, Walton Manner, Milton Keynes, Bucks MK7 7AJ, United Kingdom. Particularly preferred are Celanex® 2500 and Celanex® X 500/2.

In another preferred embodiment of the present invention, the dispensing device has a bespoke mouthpiece, the mouthpiece being adapted to channel and direct the composition according to the first aspect of the invention from an orifice of the device, towards the sublingual area of the patient. Such a mouthpiece could be used in conjunction with a conventional spray device, such as one of the types discussed above.

Preferably, the mouthpiece of the dispensing device is angled in relation to the main body of the device, so that the mouthpiece directs the dispensed composition to the sublingual mucosa when the device is activated whilst held in the normal position for use.

Such a mouthpiece could be used in conjunction with devices having either an upright or an inverted valve. In a preferred embodiment, the device has an inverted valve, such devices generally being capable of dispensing accurate volumes of composition.

In a further preferred embodiment, the mouthpiece for directing the dispensed composition to the sublingual area is part of a housing in which the main body, including the container, of the spray device is held. The mouthpiece could be rigidly fixed with respect to the housing, or the connection between the housing and the mouthpiece could be flexible, allowing the angle of the mouthpiece relative to the main body of the device to be adjusted.

In a further embodiment, the mouthpiece is shaped in such a way that it assists directional dispensing of the composition to the sublingual area of the mouth.

Preferably, the mouthpiece is long enough to allow the opening of the mouthpiece to sit under the tongue when the composition is dispensed. This will reduce the amount of composition being dispensed to parts of the oral cavity other than the sublingual area. Even more preferably, for greater comfort and greater ease of use, the mouthpiece is also a slim shape, fitting comfortably under the tongue or being comfortably held to direct the spray towards the sublingual area.

Additionally, the mouthpiece may also be shaped in such a way that it discourages the spread of the dispensed composition after it leaves the mouthpiece. When a composition is dispensed by a conventional spray device it will generally spread, forming a cloud. This is undesirable where a small area of the oral cavity is to be targeted. In a preferred embodiment, the mouthpiece opening is no larger than the average size of the sublingual area. This means that, despite some degree of spreading of the dispensed composition after it has left the mouthpiece, the spread will be limited to ensure that the area of the oral cavity contacted by the dispensed composition will correspond generally to the sublingual area, provided the composition is dispensed in the correct direction.

It is also advantageous for the dispensing device to be adapted to reduce or control the velocity at which the dispensed composition leaves the device. This will help to ensure that the composition comes into contact with the sublingual mucosa and stays in contact for long enough for the pharmaceutically active agent to be absorbed. Such control may be provided, to an extent, by the shape of the mouthpiece of the dispensing device.

Thus, in a possible embodiment, the mouthpiece of the spray device has a cross-sectional area which first gradually increases, and then decreases. The resultant “duckbill” shape may both control the velocity of the dispensed composition and limit its spread. It is clear that a variety of mouthpiece shapes may be used to reduce the velocity of the dispensed composition.

In another preferred embodiment, the velocity with which the composition is dispensed is also reduced by providing the device with a plurality of orifices through which the composition is released. The provision of more than one orifice will reduce the force with which the dispensed composition is released from the main body of the device, thereby reducing its exit velocity. The more orifices through which the composition is dispensed, the slower the velocity of the substance dispensed.

In a yet more preferred embodiment, in a device having a plurality of orifices these orifices may be shaped and positioned to be directional, preferably serving to direct the individual jets of dispensed composition toward on another, to avoid unnecessary and undesirable spreading of the composition around the oral cavity.

Most preferably, the orifices are directed so that jets of dispensed composition converge at a point which is approximately the same distance from the nozzle of the device as the sublingual area will be from the nozzle when the device is used. Thus, the composition should contact a relatively small area, avoiding wastage caused by the composition being dispensed to areas other than the sublingual mucosa.

In a preferred embodiment of the invention, the orifices of the device are further adapted to dispense particles of a particular size, thereby optimising absorption across the sublingual mucosa.

Some examples of devices for sublingual delivery of opioid analgesics will now be described, by way of example only, and with reference to the following drawings.

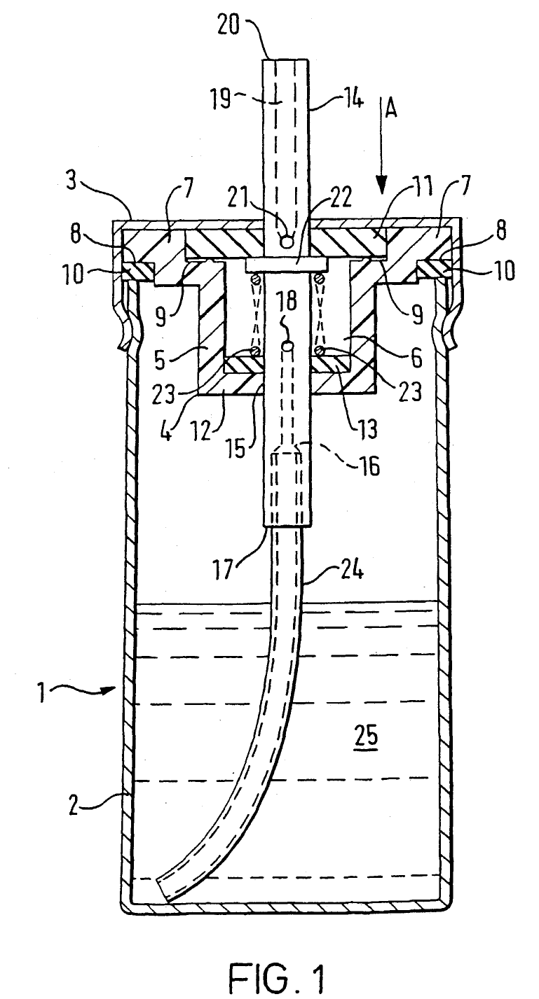

FIG. 1 is a cross sectional view of an embodiment of a device in accordance with the invention.

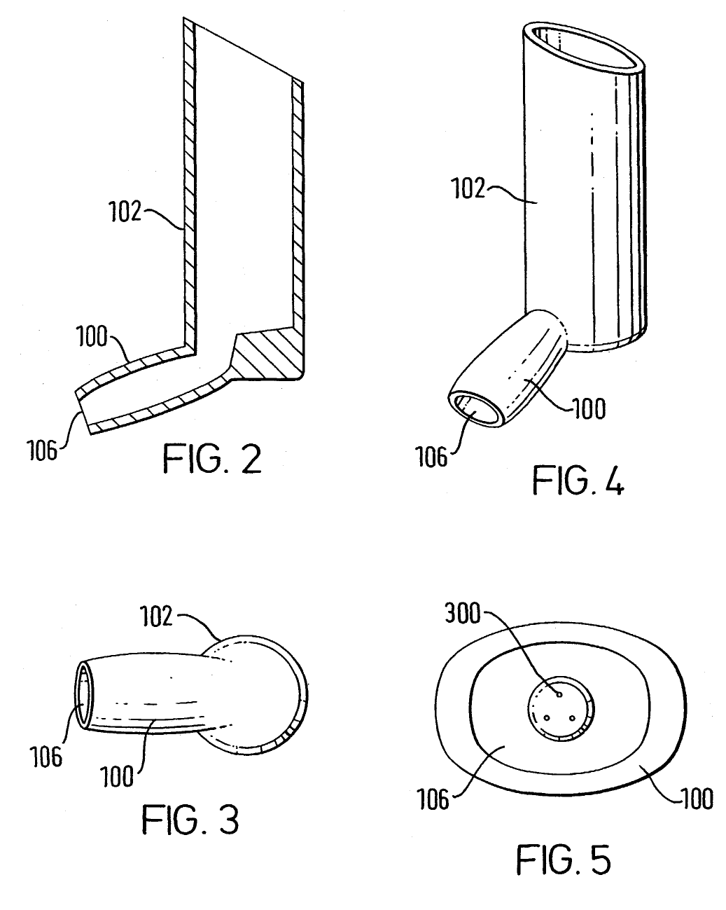

FIG. 2 is a cross sectional view of an embodiment of a housing, including a mouthpiece, for an inverted valve device, in accordance with the present invention.

FIG. 3 is a view of the underside of the housing shown in FIG. 2.

FIG. 4 is a perspective view of the housing shown in FIG. 2.

FIG. 5 is a view down the mouthpiece of the housing shown in FIG. 2, the housing containing a spray device with a nozzle having three orifices.

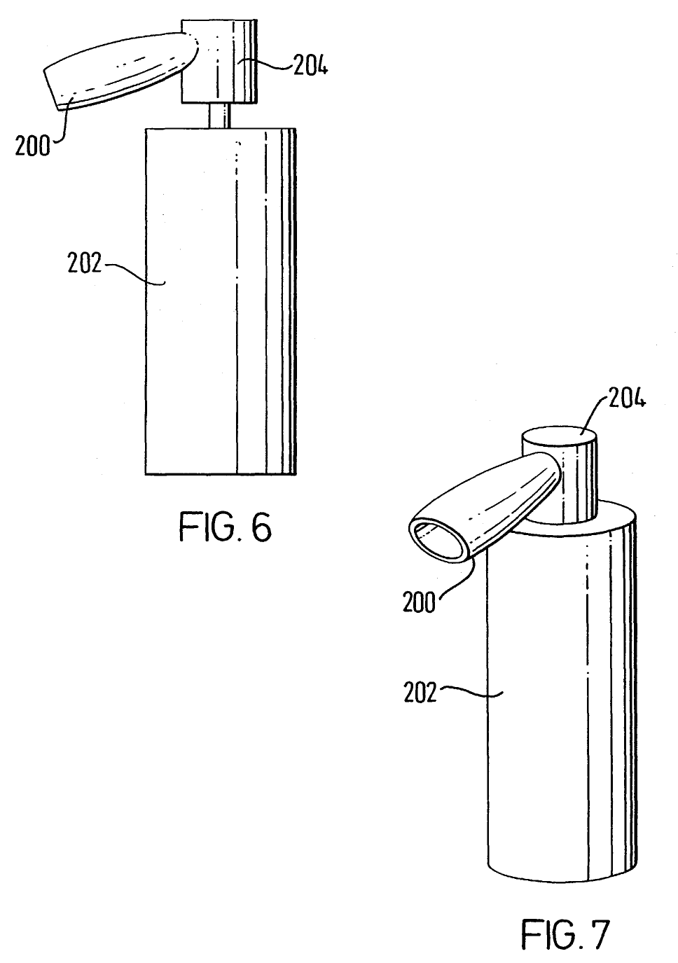

FIG. 6 is a side view of a device with an upright valve, and a mouthpiece according to the present invention.

FIG. 7 is a perspective view of the device shown in FIG. 6, wherein the spray device is being activated by downward pressure on the top of the device.

The device 1, shown in FIG. 1, comprises a substantially cylindrical canister 2 sealed with a cap 3. Both the canister 2 and the cap 3 may be manufactured from a variety of materials. The canister and cap may be lined with a polyester (such as Celanex® 2500) or a lacquer (not shown).

A valve body moulding 4 comprises a cylindrical portion 5, which defines a metering chamber 6 and a stepped flange portion 7, and is formed by injection moulding from Celanex® 2500. The stepped flange portion 7 defines a first and outwardly facing annular seat 8 and a second, inwardly facing annular seat 9. The first annular seat 8 accommodates an annular sealing ring 10 and the second annular seat 9 accommodates a first sealing washer 11. The first sealing washer 11 is located so as to cooperate with the cylindrical portion 5 of the valve body moulding 4, in defining the metering chamber 6.

A base 12 of the cylindrical portion 5 of the valve body moulding 4 completes the boundary to the metering chamber 6 and provides a seat for a second sealing washer 13.

The sealing ring 10 and the first and second sealing washers 11 and 13 can be formed from a butyl rubber, neoprene or one of the elastomers disclosed for such purposes in WO 92/11190.

An elongate, substantially cylindrical and partially hollow valve core 14 is slidably located within the first and second sealing washers 11 and 13 and extends through an orifice 15, defined in the base 12. The valve core 14 is formed by injection moulding from Celanex® 2500.

A stepped inlet passage 16 communicates between a first end 17 of the valve core 14 and an inlet orifice 18, formed through the side of the valve core 14. In a likewise manner, an outlet passage 19 communicates between the second end 20 of the valve core 14 and an outlet orifice 21 formed through the side of the valve core 14. An annular flange 22 extends radially outwardly from the valve core 14 between the inlet and outlet orifices 18 and 21 and adjacent to the outlet orifice 21.

A stainless steel compression coil spring 23 acts between the annular flange 22 and the second sealing washer 13, urging the annular flange 22 into contact with the first sealing washer 11, such that the outlet orifice 21 lies inside the first sealing washer 11 and is thereby isolated from the metering chamber 6. In this position, as shown in FIG. 1, the inlet orifice 18 is located within the metering chamber 6. A flexible tube 24 is engaged within the stepped inlet passage 16 and extends from the valve core 14 to the base of the canister 2 (as shown in FIG. 1). Thus, the inlet orifice 18 is in communication with a region within the canister 2 adjacent to its base 12.

The cap 3 is firmly attached to the canister 2 by crimping and, thus, holds the assembly of the valve body moulding 4, valve core 14, coil spring 23, sealing washers 11 and 13 and sealing ring 10 in place as shown in FIG. 1, with the sealing ring 10 and first sealing washer 11 sufficiently compressed to seal the interior of the device 1 and prevent the egress of its contents.

Downward movement of the valve core, in the direction of arrow A, against the action of the spring 22 will bring the outlet orifice 21 into the metering chamber immediately after the first orifice 18 has been sealed from the metering chamber 6 by the second sealing washer 13.

When filled with a composition in accordance with the present invention, as shown at 25, the device 1 will provide metered doses of the composition when used as follows. The device 1 should be held in the position shown in FIG. 1, so that the composition 25, by virtue of its pressure, enters the metering chamber 6 via the tube 24, the inlet passage 16 and the inlet orifice 18. Subsequent depression of the valve core 14, in the direction of arrow A, seals the inlet orifice 18 and hence the remainder of the canister 2, from the metering chamber 6 and opens the outlet passage to the metering chamber 6, via the outlet orifice 21. Since the composition 25 in the metering chamber 6 is pressurised with the propellant, it will be expelled from the metering chamber 6 through the outlet orifice 21 and the outlet passage 19. If the valve core 14 is then allowed to return to the position shown in FIG. 1, under the influence of the spring 22, the outlet orifice 21 is again sealed from the metering chamber 6 and the metering chamber 6 will be filled with pressurised composition 25 from the canister 2, via the tube 24, stepped inlet passage 16 and inlet orifice 18.

Whilst the foregoing description relates to a device having an upright valve, it is clear that devices with inverted valves may also be used to dispense the compositions of the present invention. Typical suppliers of inverted valves include Bespak plc, King's Lynn, UK; 3M Neotechnic, Clitheroe, UK; and Valois Pharm, Le Vaudreuil, France.

FIGS. 2 to 7 show a mouthpiece according to a preferred embodiment of the present invention. In FIGS. 2 to 5, the mouthpiece 100 is illustrated as being part of the housing 102 which is used to house an inverted valve spray device. In FIGS. 6 and 7, the mouthpiece 200 is used with a conventional upright valve device 202 and is affixed to the moveable button part 204 of the dispensing device. Thus, when the device is in use, the moveable button 204 is depressed and the mouthpiece will also move relative to the main body of the dispensing device.

Upon activating either type of spray device illustrated in the figures, the composition held within the container is dispensed from the spray device. As it leaves the spray device, the dispensed composition enters the mouthpiece. The mouthpiece then channels the composition to the opening of the mouthpiece 106.

In use, the mouthpiece is preferably placed under the tongue, with the opening of the mouthpiece adjacent to the sublingual area. This ensures that the dispensed composition contacts, almost exclusively, the sublingual area when it leaves the mouthpiece.

The figures illustrate the preferred shape of the mouthpiece. The mouthpiece has a smooth shape, with a gradually increasing cross-sectional area which then decreases again towards the opening.

In FIG. 5, the orifices 300 of the dispensing device are shown. There are three orifices, and each is directional, so that the jets of composition dispensed therefrom converge at a predetermined distance from the outlets themselves.

There now follow some examples of compositions according to the present invention for sublingual delivery.

### EXAMPLE 1

A composition comprising fentanyl free base with HFC-134a suitable for use in a device as described above can be formulated from the following ingredients:

| Component | percent w/w | g/can |
|---|---|---|
| Fentanyl free base | 0.7 | 0.099 |
| Ethanol 96% BP | 13.2 | 1.866 |
| Peppermint oil | 1.4 | 0.205 |
| HFC-134a | 84.7 | 12.02 |
| Total | 100 | 14.19 |

The peppermint oil is added to the fentanyl/ethanol solution and mixed thoroughly. 2.17 g of the resulting solution is then placed in the canister 2 and the valve assembly, comprising the valve body moulding 4, first sealing washer 11, second sealing washer 13, spring 22, tube 23, and annular seal 10 are then sealed onto the canister 2 as shown in FIG. 1 by the cap 3. The propellant is then added to the canister by being forced through the valve core 14 at great pressure, and the complete device is then checked for leaks.

### EXAMPLE 2

A second composition comprising fentanyl free base with HFC-134a suitable for use in a device as described above can be formulated from the following ingredients:

| Component | percent w/w | g/can |
|---|---|---|
| Fentanyl free base | 0.164 | 0.010 |
| Ethanol 96% BP | 4.992 | 0.305 |
| HFC-134a | 94.844 | 5.795 |
| Total | 100 | 6.11 |

The fentanyl is dissolved in the ethanol in the proportions set out above and 0.315 g of the resulting solution is then placed in a canister 2 and a valve assembly, comprising a valve body moulding 4, first sealing washer 11, second sealing washer 13, spring 22, tube 23, and annular seal 10, is then sealed onto the canister 2 by crimping as shown in FIG. 1 by the cap 3. The propellant (HFC-134a) is then added to the canister, by being forced through the valve core 14 at great pressure, and the complete device is then checked for leaks. After the propellant entered the canister it dissolves the remaining portions of the composition.

### EXAMPLE 3

A third composition comprising fentanyl free base and suitable for use in a device as described above can be formulated from the following ingredients:

| Component | percent w/w | g/can |
|---|---|---|
| Fentanyl free base | 0.164 | 0.010 |
| Ethanol 96% BP | 7.5 | 0.458 |
| HFC-134a | 92.336 | 5.641 |
| Total | 100 | 6.11 |

The fentanyl dissolved in the ethanol in the proportions set out above and 0.315 g of the resulting solution is then placed in a canister 2. A valve assembly (as described in Example 2) is then sealed onto the canister 2 by crimping and the HFC-134a propellant is then added to the canister, by being forced through the valve core 14 at great pressure, and the complete device is then checked for leaks. After the propellant entered the canister it dissolves the remaining portions of the composition.

### EXAMPLE 4

Further compositions comprising fentanyl free base with HFC-134a, suitable for use in a device as described herein, can be formulated in accordance with the details set out in the following table, in which all figures are given on a percent by weight basis.

| Composition | A | B | C | D | E |
|---|---|---|---|---|---|
| Fentanyl free base | 0.164 | 0.164 | 0.164 | 0.164 | 0.164 |
| Transcutol ® | 9.984 | 4.992 | | | |
| Oleyl alcohol | | | 2.496 | | |
| Propylene glycol | | | | 4.992 | |
| Ethanol | | 4.992 | 7.488 | 4.992 | 20.51 |
| HFC-134a | 89.852 | 89.852 | 89.852 | 89.852 | 79.326 |
| Total | 100 | 100 | 100 | 100 | 100 |

Compositions A-E are prepared using a similar technique to that set out in Example 2 above. Briefly, the fentanyl and other excipient or excipients (excepting the HFC-134a) are mixed and placed in a canister 2. A valve assembly is then sealed onto the canister 2 by crimping and the HFC-134a propellant is then added to the canister 2, by being forced through the valve core 14 at great pressure. After the propellant enters the canister 2, it dissolves the remaining portions of each composition.

Although only fentanyl free base is referred to in the above mentioned examples, other forms of fentanyl and other opioid analgesics previously discussed in this application may be substituted therefor in quantities which would dissolve in the propellant or in the other proposed composition constituents.

1. A pharmaceutical composition comprising a solution of an opioid analgesic and a propellant, for sublingual aerosol administration.

2. A pharmaceutical composition as claimed in claim 1, wherein the opioid analgesic is selected from fentanyl, alfentanil, carfentanil, remifentanil, sufentanil, buprenorphine, morphine, and diamorphine.

3. A pharmaceutical composition as claimed in claim 1 or 2, wherein the opioid analgesic is in the free base form.

4. A pharmaceutical composition as claimed in claim 3, wherein the opioid analgesic is fentanyl free base.

5. A pharmaceutical composition as claimed in any of the preceding claims, wherein the solution is substantially free of water.

6. A pharmaceutical composition as claimed in claim 5, wherein the concentration of water is no more than 0.25%, and is preferably no more than 0.15%.

7. A pharmaceutical composition as claimed in any of the preceding claims, wherein the propellant is 1,1,1,2-tetrafluoroethane HFC-134a) or 1,1,1,2,3,3,3-heptafluoropropane (HFC-227), or a combination thereof.

8. A pharmaceutical composition as claimed in claim 7, comprising HFC-134a as the propellant.

9. A pharmaceutical composition as claimed in claim 7, comprising between 50 and 99% w/w and, preferably, between 75 and 95% w/w HFC-134a or HFC-227.

10. A pharmaceutical composition as claimed in any of the preceding claims, further comprising a carrier selected from pharmaceutically acceptable alcohols, polyols, (poly) alkoxy derivatives, fatty acid alkyl esters, polyalkylene glycols, and dimethyl sulphoxide.

11. A pharmaceutical composition as claimed in claim 10, wherein the carrier is ethanol.

12. A pharmaceutical composition as claimed in claim 10 or 11, comprising up to 50% w/w, preferably up to 25% w/w carrier.

13. A pharmaceutical composition as claimed in any of claims 10-12, comprising a plurality of carriers.

14. A pharmaceutical composition as claimed in any of the preceding claims, further comprising an organic surfactant.

15. A pharmaceutical composition as claimed in claim 14, wherein the surfactant is oleyl alcohol.

16. A pharmaceutical composition as claimed in any of the preceding claims, wherein the composition is substantially free of weak organic or strong inorganic acid.

17. A pharmaceutical composition as claimed in any of the preceding claims, further comprising a flavouring oil, preferably peppermint oil.

18. A pharmaceutical composition as claimed in claim 1, comprising HFC-134a, fentanyl free base and ethanol.

19. A pharmaceutical composition as claimed in any of the preceding claims, comprising a further pharmaceutically active agent.

20. Use of an opioid analgesic in the manufacture of a medicament for sublingual aerosol administration, the medicament comprising a pharmaceutical composition as claimed in any of claims 1-19.

21. A device for providing pharmaceutical doses comprising a container filled with a pharmaceutical composition according to any of claims 1-19 and valve means arranged for delivering aerosol doses of said pharmaceutical composition to the exterior of the container.

22. A device as claimed in claim 21, wherein at least a portion of the device is formed from a polyester.

23. A device as claimed in claim 21 or 22, wherein the valve means includes at least one component formed from a polyester.

24. Use of a polyester in contact with a composition as defined in any of claims 1-19.

25. A mouthpiece for use with a spray device for dispensing a composition defined in any one of claims 1-19 to the sublingual area of the oral cavity, the mouthpiece being shaped to facilitate dispensing said composition to the sublingual area.

26. A mouthpiece as claimed in claim 25, wherein the mouthpiece is angled with respect to the main body of the spray device so that, upon activation of the device, the mouthpiece will be directed towards the sublingual area.

27. A mouthpiece as claimed in either of claims 25 and 26, wherein the mouthpiece is shaped to comfortably lie beneath the tongue.

28. A mouthpiece as claimed in any one of claims 25-27, wherein the cross-sectional shape of the mouthpiece is generally an ellipse.

29. A mouthpiece as claimed in any one of claims 25-28, wherein the mouthpiece is shaped to reduce the extent to which the composition to be dispensed disperses upon leaving the mouthpiece.

30. A mouthpiece as claimed in any one of claims 25-29, wherein the mouthpiece is shaped to slow the velocity of the composition dispensed by the spray device.

31. A housing for a container of a spray device, the housing including a mouthpiece as claimed in any of claims 25-30.

32. A device as claimed in any one of claims 21-23, the device including a mouthpiece as defined in any one of claims 25-30.

33. A device as claimed in any one of claims 21-23, the device including a housing as claimed in claim 32.

34. A device as claimed in any one of claims 21-23, 32 and 33, wherein the composition is dispensed into the mouthpiece through a plurality of orifices.

35. A device as claimed in claim 34, wherein the orifices direct the dispensed composition.

36. A device as claimed in claim 35, wherein the composition dispensed through each of the plurality of orifices converges.

37. A device as claimed in claim 36, wherein the dispensed composition converges approximately at the sublingual mucosa when the device is in use.

38. A device, substantially as hereinbefore described and as shown in the accompanying drawings.

39. A mouthpiece, substantially as hereinbefore described and as shown in the accompanying drawings.

40. A housing for a device, substantially as hereinbefore described and as shown in the accompanying drawings.
